(19) United States
(12) Patent Application Publication
(10) Pub. No.: US 2026/0112290 A1
(43) Pub. Date: Apr. 23, 2026

(54) Wearable Device for Supporting Behavioral Self Management

(71) Applicants: Brian Jeffrey Fogg, Healdsburg, CA (US); Riley Armstrong Fogg, Healdsburg, CA (US)

(72) Inventors: Brian Jeffrey Fogg, Healdsburg, CA (US); Riley Armstrong Fogg, Healdsburg, CA (US)

(21) Appl. No.: 18/924,917

(22) Filed: Oct. 23, 2024

(51) Int. Cl. G09B 19/00; A44C 5/00; A61B 5/00

(52) U.S. Cl. CPC G09B 19/00; A44C 5/0015; A61B 5/486; A61B 5/681

(57) ABSTRACT

A wearable device for improving outcomes of behavioral self-management having message plate or tag having two sides, wherein each of the sides is suitable for receiving at least a first indicia and a second indicia, and a retention strip attached to the message plate or tag maintains the message plate or tag in at least two positions, each position revealing one user-selected indicia and concealing a user non-selected indicia. The wearable device may be an accessory for a bracelet, watch band, or other user-worn piece of clothing or jewelry, or it may be a wearable device in and of itself. It is suitable for use in creating reminders for the user, and especially for use in behavioral self-management.

### INCORPORATION BY REFERENCE

The published book entitled “Tiny Habits: The Small Changes That Change Everything” by B. J. Fogg, Ph. D. (Houghton Mifflin Harcort (2020), New York, New York) and cataloged by the Library of Congress as LCCN 2019023920 (ISBN 9780358003328) is hereby incorporated by reference in its entirety, and all copyrights are reserved.

### FIELD OF THE INVENTION

The present invention relates to wearable devices, jewelry, clothing, and fashion accessories for promoting personal improvement through Self Behavioral Management methods and techniques.

### BACKGROUND OF THE INVENTION

Behavioral Self Management (BSM) and its related Behavioral Modification Therapy (BMT) are both well proven methodologies for changing a person's behavior through intentional management of cognitive processes, cues, responses and consequences. The former tends to be more self-guided through books, videos, and online literature, while the latter is usually considered to be led by a trained and licensed professional such as a therapist.

According to the American Psychological Association (APA) Dictionary of Psychology, self-management is (n.):
1. an individual's control of their behavior, particularly regarding the pursuit of a specific objective (e.g., weight loss). Self-management is usually considered a desirable aspect for the individual personally and socially, but some forms of self-management may be detrimental to mental and physical health. Psychotherapists and counselors often seek to provide clients with methods of identifying the latter and modifying them into the former.
2. a behavior therapy program in which clients are trained to apply techniques that will help them modify an undesirable behavior, such as smoking, excessive eating, or aggressive outbursts. Clients learn to pinpoint the problem, set realistic goals for changing it, use various contingencies to establish and maintain a desired behavior, and monitor progress.

Behavioral Self-Management (sometimes written without the hyphen) is the set of theories, processes, methods, science and research in which individuals are empowered with various techniques and sometimes supporting devices to observe their own reactions, in real time or in post-analysis, to stimuli, events, situations, etc., and how they responded, and to intervene in their own response process to intentionally change their response from that of autonomic or habitual to intentional. This can promote reduction or elimination of unwanted, hurtful, self-defeating or inappropriate autonomic habitual responses, with optional forming of new autonomic or habitual responses that are wanted, helpful, self-benefiting and appropriate.

### SUMMARY OF THE INVENTION

A wearable device and method for improving outcomes of behavioral self-management are disclosed, wherein the device has flippable or rotatable message plate or tag having two sides, wherein each of the sides is suitable for receiving at least a first indicia and a second indicia, and having a retention strip attached to the message plate or tag maintains the message plate or tag in at least two positions, each position revealing one user-selected indicia and concealing a user non-selected indicia. The wearable device may be an accessory for a bracelet, watch band, or other user-worn piece of clothing or jewelry, or it may be a wearable device in and of itself. It is suitable for use in creating reminders for the user, and especially for use in behavioral self-management.

### BRIEF DESCRIPTION OF DRAWINGS

The figures presented herein, when considered in light of this description, form a complete disclosure of one or more embodiments of the invention, wherein like reference numbers in the figures represent similar or same elements or steps.

FIG. 1 shows a perspective view of an example manner of usage of an embodiment according to the present invention, in a first position or configuration, thereby revealing a first message to a viewer.

FIG. 2 shows a first or front side view of an example embodiment according to the present invention, revealing a first message to a viewer.

FIG. 3 illustrates a second or back (reverse) side of an example embodiment according to the present invention, revealing a second message to a viewer.

FIG. 4 provides a straight on view in greater detail of the example embodiment of FIG. 1.

FIG. 5 provides a straight on view in greater detail of the example embodiment of FIG. 1 from the inside of a watch band, bracelet or wrist band.

FIG. 6 depicts a side view of an example embodiment of the present invention in a first position with the first (front) side facing away from the watch band, bracelet or wrist band.

FIG. 7 depicts a side view of an example embodiment of the present invention in a first position in transition from the first (front) side facing away from the watch band, bracelet or wrist band, towards a second position in which a second (back) side of the example embodiment will be revealed.

FIG. 8 illustrates a completion of the transition motion of FIG. 7.

FIG. 9 depicts a side view of an example embodiment of the present invention in a second position with the second (back) side facing away from the watch band, bracelet or wrist band, thereby revealing a second message to a viewer.

FIG. 10 illustrates a perspective view of an example manner of usage of an embodiment according to the present invention, in a second position or configuration, thereby revealing a second message to a viewer.

FIG. 11 shows a feature of at least one embodiment to provide a tactile feedback to the user.

### DETAILED DESCRIPTION

The present inventors have improved upon the known structures, mechanisms and design of wearable devices, jewelry, clothing, and fashion accessories for promoting personal improvement through Behavioral Self Management (BSM) methods and techniques. Various embodiments include, but not limited to, accessory devices attachable to a watch band, bracelet, or wristband, as well a separate watch bands, bracelets and wristbands incorporating the elements and functions of the invention. The following detailed description of an embodiment in the form of an accessory for a watchband is just one example embodiment, and does not represent the full scope and range of embodiments according to the present invention.

One of the present co-inventors is an acclaimed author of a book on Behavioral Self Management, and the founder of a behavioral science lab at a major university. In his work with clients wishing to reduce or eliminate targeted habits, reflexive behaviors and autonomous responses to stimulus, events, and provocations, as well as clients wishing to learn new habits, he has discovered that many of the wearable items fall short of the needs in the art. There are number of bracelets, beads, tags and even electronic wrist-worn items which purport to help with BSM, none of them bring the following elements to the process.

To the best of the co-inventors' knowledge, there aren't many technologies available for self-reinforcement behavior modification. Some wearable devices provide a level of self-reinforcement, such as certain smart watches, but these wearable technology devices can only reinforce behaviors that they can sense biologically or biometrically, and they cannot reinforce thoughts/mantras, subtle movements, or words used in a conversation. So, at least one objective of the present invention is to provide self-reinforcement for these kinds of habits, actions and self control behaviors without requiring biological or biometric sensing.

Additionally, timing of the self-reinforcement is important. Smart watches can be slow to provide reinforcement messages, buzzes or tones. For example, a smart watch may congratulate you AFTER a workout is completed, which is not optimal for habit formation. Therefore, at least another objective of the present invention is to provide optimal timing of the reinforcement at the beginning of the desired behavior, such as an exercise workout.

For example, “clicker training” is when a trainer (a person, coach, etc.) clicks at the exact moment the subject, such as an athlete, starts to do the target behavior. Clicker training is a powerful system because the click immediately signals to the subject's brain precisely what is the desired behavior, which is believed to provide an dopamine release in the optimal moment needed to cause a new habit to form. It is believed that a reinforcement that comes even 5 seconds later won't cause a release of dopamine in the best way to cause the habit to form. As such, at least another objective of some embodiments of the present invention is to provide precise timing of the reinforcement message, touch, etc., so the user's brain can immediately understand the cause and effect relationship, thereby allowing the target behavior to lead to this positive result (feeling of success) as a result of an optimally-timed dopamine release.

Still further, all of the wearables of which the present inventors are aware only provide one message of encouragement, or one message of a goal or objective, such as “Will Power” as it appears in U.S. pre-grant published patent application No. 200800060116A1 to Leon Massage. This falls short of providing a second message in the form of positive feedback or encouragement when the goal or objective of the first message is met or performed. Without this second message, the habit of the first message is not reduced or instilled, it just serves as a reminder and is likely to become forgotten over time. Therefore, it is another objective of some embodiments of the present invention to remedy this unmet need in the art to provide a second, positive reinforcement message.

Another objective of some embodiments of the present invention is to provide a mechanism for recording the achievement throughout the rest of the day so that, at the end of the day when the wearer removes the wearable item, they are reminded as second time of having met their goal or objective. This doubles the beneficial affect of the positive reinforcement of the second message, increasing rate at which the unwanted habit is reduced, and/or increasing the rate at which a new desirable habit is formed.

Still another objective of some embodiments of the present invention is to provide a tactile feedback which increases neurological feedback associated with the reception of the second message of positive reinforcement. This neurological feedback promotes even quicker reduction of an unwanted habit and/or quicker learning of a new habit.

These and other objectives will be apparent throughout the following paragraphs. Some embodiments may meet fewer than all of these objectives, while still providing improvements over the devices presently available for BSM.

Terminology. For the remainder of this disclosure, we will refer to the first message as a “target behavior”, which includes but is not limited to targeted (desired) thoughts, mantras, subtle movements, an encouragement, a goal, an objective and words used in a conversation, whether or not they are part of a formal Behavior Self-Management program or process. We will also refer to the second message as a “reinforcement”, which includes but is not limited to a positive message acknowledging the completion of the target behavior and a tactile feedback feature such as a bump or ridge.

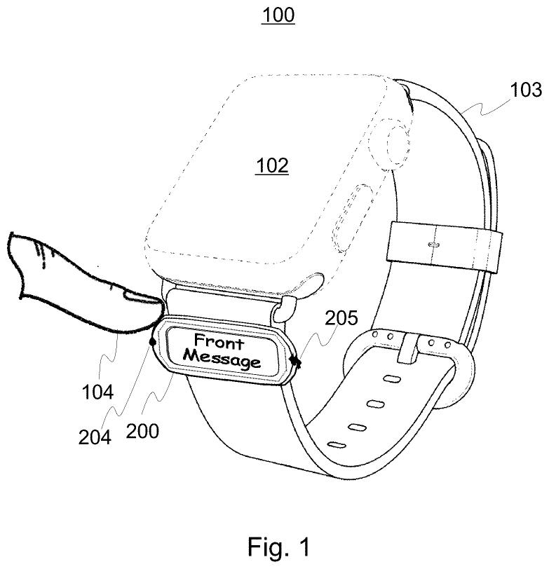

Example Structural Embodiments. Referring now to FIG. 1, a configuration of elements is shown 100 in which an embodiment of the invention is attached to a typical band 103 of a wrist watch 102. The embodiment may take the form of a relatively flat plate or tag, such as a rectangle or an oval, about the width of the watch band, and having a first side with a first message 200 regarding a target behavior. In this example embodiment, the plate or tag is attached to the watch band 103 by a section of elastic cord tethered or attached 204, 205, at opposite edges of the plate or tag, and extending behind the strap 103 such that the strap is captured between a second side (not visible in FIG. 1) of the tag or plate and the elastic cord section. Other embodiments may use a thin strip of other material, such as rubber, neoprene, silicone, leather, chain, natural fibers, etc. It will be apparent in the following paragraphs some mechanical requirement of this attachment to support proper operation of the device as an BSM assistive tool.

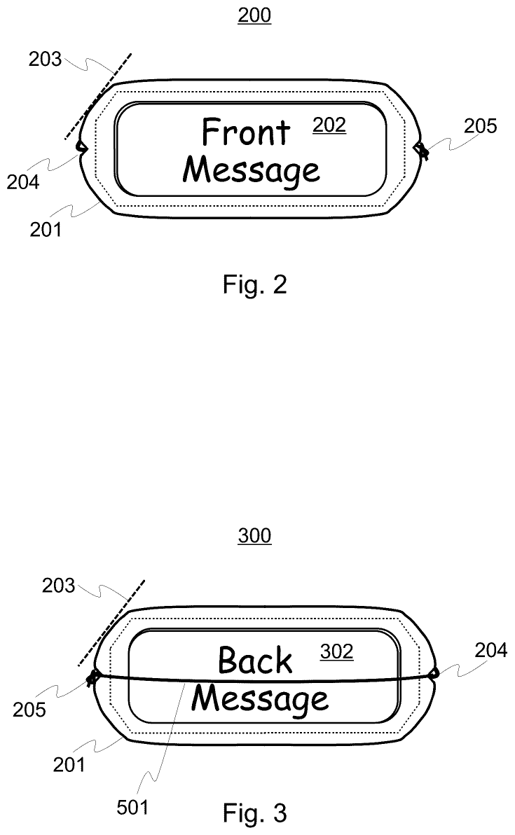

Referring now to FIG. 2, a front side 200 having a first message indicia embossed, debossed, printed, etched, written, or coded (such as braille), such as a label received into a recess 202. This indicia may be pre-determined, such as a manufactured message, or it may be user-determined such as a hand-written or user printed message on a label. In this example embodiment, the edges of the label are protected by the raised lips around the recess such that it will not be easily scraped or scratched off during wear by a user, and there are two attachment points 204, 205, for the retainer strip, such as recesses and holes through which elastic cord can be received and tied. Other embodiments may use other types of affixed indicia that labels, and may use other types of retainer strip such as rubber bands, silicone straps, etc. A benefit of the label-based embodiment is that, once a first goal or objective has been met multiple times or the message of encouragement has been fully taken to heart, the label can be removed and a new label with a new first message applied.

FIG. 3 shows the same example embodiment of FIG. 2 except as viewed from behind the watch strap, IE., the wearer's skin side of the watch strap. This second side 300 of the plate or tag is mechanically essentially the same as the first side 200 with a means for receiving and retaining a second message, wherein the second message on the second side 300 is a reinforcement to the target behavior first message on the first side 200.

Also visible in FIGS. 2 and 3 is a preferred angled or rounded shape 203 of the corners of the plate or tag to promote smoother operation of the device, as will be discussed in the following paragraphs.

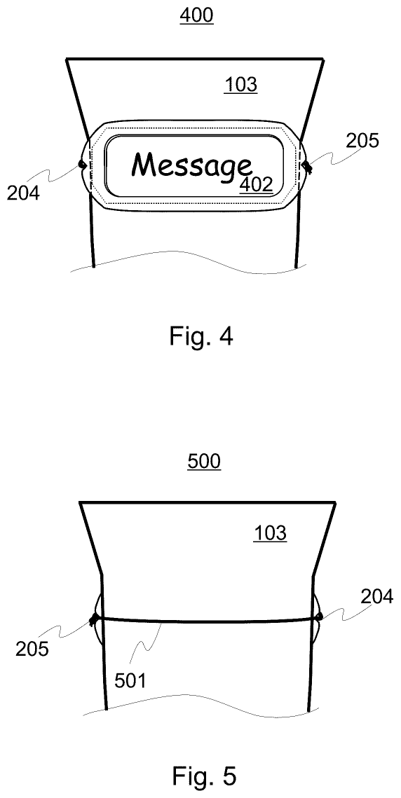

Referring now to FIG. 4, a straight-on view 400 of the exposed side and message 402 of the device on a portion of a watch band 103 is shown, and the reverse, skin side of the band is shown 500 in FIG. 5, including the retainer strip 501, as previously described with respect to this example embodiment.

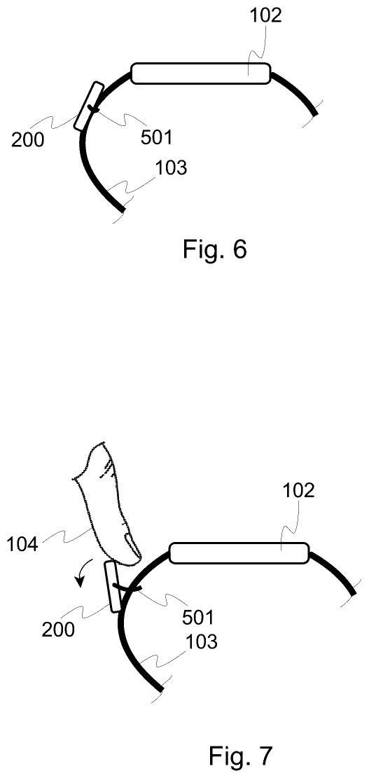

Manner of Operation and Use. In order to form a target behavior or to reduce an existing target behavior, such as but not limited to a practice of BSM, the device would be prepared or provided with two related messages: a first message regarding the target behavior and a second message of reinforcement for beginning the target behavior. The device of the foregoing example embodiment would be then slid onto a watch band, bracelet or wrist band 103 as shown in FIG. 6 from a side view, with the first message side showing 200, such as at the beginning of a day, beginning of a social event or a beginning of a work shift. This provides for a visible reminder to the wearer of the goal, objective or encouragement, and can provide as a “fiddle” object to touch when feel an undesired habit or response beginning, such as an anxiety in response to a certain situation (getting on a plane, meeting new people, answering questions, etc.).

After the wearer has successfully started the target behavior, such as avoiding panic breathing when boarding an airplane, avoiding meeting people at a social engagement, willingly engaging in answering questions, etc., the user may initiate flipping the tag or plate with one's finger 104, as shown in FIG. 7. Please note that this diagram begins to show one of the operational and structural features of embodiments of the invention in that it securely holds the tag or plate in the first position, but also allows the tag or plate to be flipped without excessive operational steps like removing the tag from the band or unlatching it, etc. This embodiment simply allows the elastic cord of the retainer strip 501 to stretch to temporarily accommodate the new, partially rotated tag or plate.

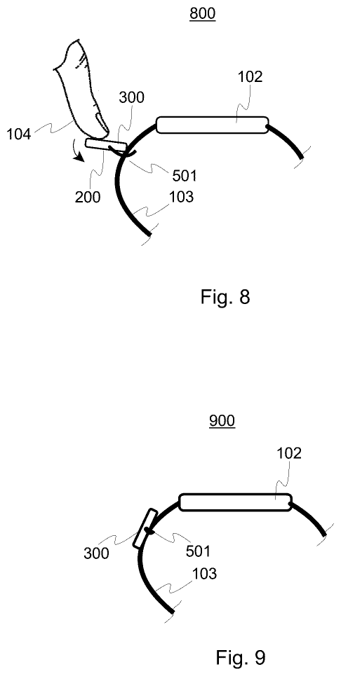

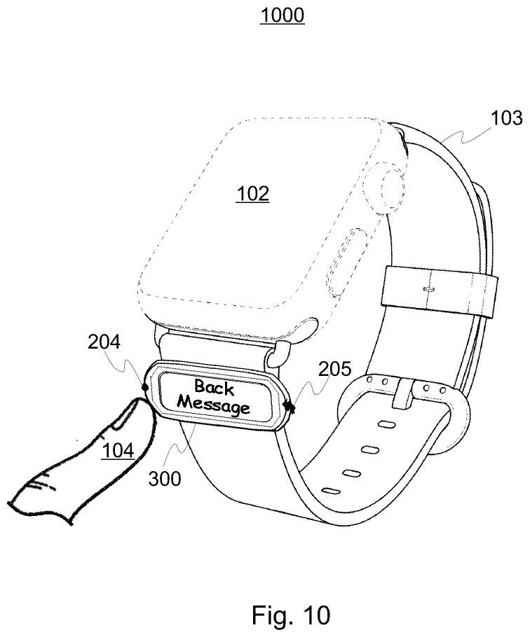

Referring now to FIGS. 8 and 9, it can be seen that the flipping operation 800 is completed 900 when the tag reaches a second position in which the second message on the second side 300 is revealed, and the first message is concealed against the band 103. The retainer strip now keeps the tag or plate securely in this second position so that it acts as a reminder for the rest of the wear period, such as the rest of the day, rest of the work shift, rest of the social event, rest of the flight, etc., of the user's accomplishment. And, because it conceals the related first message, as shown 1000 in FIG. 10, the user's privacy of their self-behavior modification process is protected should an onlooker see the second message.

Second Example Method of Use. In another example method of use, the first message of a targeted behavior disposed on the first side 200 may require the user to complete an affirmative action to begin to instill a new habit, response, etc., in response to the situation, stimulus, etc., before achieving or earning the second message on the second side. For example, if the goal is to overcome anxiety to meeting new people and to quit dodging introductions at meetings, parties, etc., the required action may be to actually initiate an introduction by selecting someone in the room to introduce themselves to. The Table below shows some example target behavior messages and corresponding reinforcement messages.

TABLE
Example Target Behavior and Reinforcement Message Pairs

| Target Behavior Message | Reinforcement Message |
|---|---|
| Drink some water | Hooray! Hydration matters |
| Eat a healthy snack | Good for me! |
| Take a break when needed | Excellent |

As shown in the Table, a first message may say, “Drink some water”, and a second corresponding message might say, “Hooray! Hydration Matters.”

Improved Flip Capability and Position Retention. As previously mentioned, at least in one embodiment, corners of the plate or tag that carries the messages are rounded or angled 203. This improves the ability of the stretchable retention strip, such as a portion of elastic cord, to pass over the surface of the plate or tag and around the corners during the flipping action, such that single finger operation is reliably possible. Completing the flipping operation should be as quick and easy as possible to minimize its intrusion on the process of establishing a psychological connection between the step of achieving the goal or objective and the step of receiving the reward.

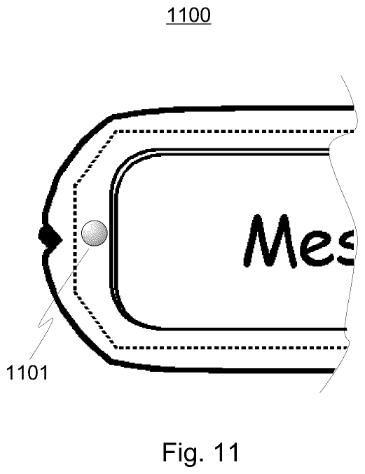

Tactile Feedback. Referring now to FIG. 11, a portion 1100 of a plate or tag according to at least one embodiment of the present invention is shown in which a tactile surface feature has been provided 1101, such as an embossed or debossed bump, ridge, shape or pattern. This feature can be, in some embodiments, added to just one side, such as the confirmation side or the goal side, or different features can be added to both sides, such as one bump on the goal side (allows the wearer to touch it without looking to remind themselves of the goal) and two bumps on the confirmation side (allows the wearer to touch it without looking at it to remind themselves of their accomplishment today, this shift, at this event, etc.). Tactile feedback increases neurological feedback associated with both the encouragement message, goal message or objective message to assist in cognitive self-monitoring and self-control, as well as increases the positive reinforcement effects of the second message, once earner or completed. This neurological feedback promotes even quicker reduction of an unwanted habit and/or quicker learning of a new habit.

Conclusion. The terminology used herein is for the purpose of describing particular exemplary embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof, unless specifically stated otherwise.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

I/We claim:

1. A wearable device for improving outcomes of behavioral self-management comprising:
a message plate having as least two sides and a periphery, each of the sides being suitable for receiving at least a first indicia and a second indicia, respectively; and
a retention strip attached at two or more points on the periphery of the message plate, wherein the retention strip maintains the message plate in at least a first position revising the first indicia and concealing the second indicia, and in at least a second position revealing the second indicia and concealing the first indicia, and wherein the position is user-selectable.

2. The wearable device as set forth in claim 1 wherein a width dimension of the message plate is between 18 mm and 22 mm, inclusive, and wherein the retention strip accommodates a watch band.

3. The wearable device as set forth in claim 1 wherein the retention strip comprises a portion of elastic cord.

4. The wearable device as set forth in claim 1 wherein the message plate further comprises a recessed area on each of at least two sides.

5. The wearable device as set forth in claim 4 wherein the recessed area matches a width and height of a corresponding label.

6. The wearable device as set forth in claim 5 further comprising:
a first label carrying the first indicia, the first label being disposed in a recessed area on a first side; and
a second label carrying the second indicia, the second label being disposed in a recessed area on a second side.

7. The wearable device as set forth in claim 1 further comprising at least a first indicia and a second indicia received on the at least two sides, respectively.

8. The wearable device as set forth in claim 7 wherein the first indicia comprises a target behavior message and wherein the at least second indicia comprises a reinforcement message.

9. The wearable device as set forth in claim 1 further comprising a tactile feature disposed on at least one of the at least two sides of the plate.

10. A method of manufacture of a wearable device for improving outcomes of behavioral self-management, comprising:
producing a message plate having as least two sides and a periphery, each of the sides being fabricated of a material suitable for receiving at least a first indicia and a second indicia, respectively; and
attaching a retention strip at two or more points on the periphery of the message plate, wherein the retention strip maintains the message plate in at least two positions, and wherein each position reveals one user-selected indicia and conceals at least one user non-selected indicia.

11. The method as set forth in claim 10 wherein a width dimension of the message plate is 18 mm to 22 mm, inclusive, and wherein the retention strip accommodates watch band.

12. The method as set forth in claim 10 wherein the attaching of the retention strip comprises attaching a portion of elastic cord.

13. The method as set forth in claim 10 wherein producing of the message plate further comprises producing a recessed area on each of at least two sides.

14. The method as set forth in claim 13 wherein the recessed area matches a width and height of a corresponding label.

15. The method as set forth in claim 14 further comprising:
disposing a first label carrying the first indicia into a recessed area on a first side; and
disposing a second label carrying the second indicia into a recessed area on a second side.

16. The method as set forth in claim 10 further comprising disposing at least a first indicia and a second indicia received on the at least two sides.

17. The method as set forth in claim 16 wherein the first indicia comprises a target behavior message and wherein the at least second indicia comprises a reinforcement message.

18. The method as set forth in claim 17 further comprising disposing one or more steps selected from the group consisting of indicia by printing, inking, painting, stamping, embossing, debossing, stenciling and labeling.

19. The method as set forth in claim 18 wherein the at least first indicia and the at least second indicia comprise a first message and a second message of a behavioral self-management message set.

20. The method as set forth in claim 10 further comprising disposing a tactile feature on at least one of the at least two sides of the plate.
